(19) United States
(12) Patent Application Publication
(10) Pub. No.: US 2024/0321447 A1
(43) Pub. Date: Sep. 26, 2024

(54) Method and System for Personalized Prediction of Infection and Sepsis

(71) Applicant: Biosigns Pte. Ltd., Singapore (SG)

(72) Inventors: Nandakumar Selvaraj, San Jose, CA (US); Michael Joseph Pettinati, Ipswich, MA (US); Milan Shah, Hopkinton, MA (US); Kuldeep Singh Rajput, Singapore (SG)

(21) Appl. No.: 17/768,178

(22) PCT Filed: Apr. 7, 2021
(86) PCT No.: PCT/SG2021/050193

§ 371 Date: Apr. 11, 2022

(51) Int. Cl. G16H 50/20; G16H 10/60; G16H 50/70

(52) U.S. Cl. CPC G16H 50/20; G16H 10/60; G16H 50/70

(57) ABSTRACT

A computation system and method for early detection of sepsis and infection in patients uses personalized detection models. The system stores patient health data for many patients, including symptoms, vital signs and disease conditions obtained from medical records and wearable/personal sensors. Multiple sepsis prediction models are then trained based on identifying cohorts of similar patients using a similarity measure or score. Each model is trained using a different cohort or similarity measure. A patient is then monitored, and the model trained on the most similar set of patients is selected to monitor the patient and identify sepsis. Further as the patient's symptoms or vital signs change, the choice of the most similar patients, and associated model, is reassessed and the most appropriate model based on the patient's current state is selected. This ensures that as the patient's symptoms change the best model is used to assess likelihood of sepsis to ensure rapid and appropriate intervention.

### CROSS-REFERENCE TO RELATED APPLICATION

This application is the United States national phase of International Application No. PCT/SG2021/050193 filed Apr. 7, 2021, the disclosure of which is hereby incorporated by reference in its entirety.

### BACKGROUND OF THE INVENTION

### Field of the Invention

The present disclosure relates to early detection of sepsis. It details a Machine Learning/Artificial Intelligence-based personalized medical system for detecting sepsis in a particular form.

### Description of Related Art

Sepsis is a clinical syndrome of physiologic, pathologic, and biochemical abnormalities induced by infection leading to life-threatening acute organ dysfunction. Infectious etiology including sepsis accounts for substantial proportions of hospitalizations, 30-day hospital readmissions and mortality rate in hospitals. A key facet of successful and cost-effective treatment of sepsis is beginning treatment as early as possible. Outcomes worsen and costs increase substantially as the infection or dysfunction of major organs progresses and patients enter septic shock. The incidences of sepsis related in-hospital deaths are substantial, and the associated healthcare costs are staggering. Marked contributors to these statistics are that patient cases were caught late.

Clinically, sepsis is recognized as a host's systemic inflammatory response syndrome (SIRS) to infection. An infection leading to organ dysfunction was termed as ‘severe sepsis’, and when sepsis induced hypotension persists despite adequate fluid resuscitation, the condition was referred to as ‘septic shock’. Recently, the Third International Consensus has updated the definitions for sepsis and septic shock conditions (sepsis-3 criteria) by removing severe sepsis condition and provided clarity for consistent use of the definitions and terminology. Accordingly, sepsis is now defined as ‘life threatening organ dysfunction caused by dysregulated host response to infection’, and its clinical diagnosis is based on acute changes in Sequential Organ Failure Assessment (SOFA) score of ≥2.

Detection of sepsis in critical and intensive care units is generally more successful than in other environments, as critical and intensive care units typically implement continuous bed-side vital sign monitoring, which when combined with laboratory testing and the clinical expertise of such units, generally succeeds in detecting sepsis and preventing worse clinical outcomes. However, outside of the critical and intensive care units (ICU), early detection of sepsis is problematic.

Firstly, outside of the critical and intensive care units patient monitoring is often quite limited and expertise in sepsis detection is more limited. Thus, patients who develop hospital acquired infections from the general wards are less likely to be recognized and have appropriate interventions enacted in the early stages of sepsis. To assist clinicians, the quick SOFA (qSOFA) score was developed based on the sepsis-3 criteria to act as a bedside tool to try and rapidly screen the patients at risk of sepsis outside of critical care. Accordingly, qSOFA measure will be positive if the patient meets 2 of 3 criteria: (i) respiratory rate of ≥22/min; (ii) altered mentation with Glagow Coma Scale of <15; and (iii) systolic blood pressure (SBP)≤100 mmHg. However, there are concerns regarding the complexity of the SOFA and qSOFA scores, the lack of clinical evidence for the validity of sepsis-3 criteria, its applicability for widespread clinical practice, and the gap in recommendations that prompt necessary measurements and laboratory tests. Furthermore, the prognostic accuracy of SIRS, SOFA, qSOFA for sepsis prediction varies widely among various retrospective clinical trials and their use appears limited for early detection of sepsis.

As it stands, the quality of sepsis detection and treatment is highly variable between hospitals. The prescribed treatment regimens at compliant hospitals are extremely rigorous and costly. Increasing the number of people suspected of sepsis erroneously (i.e. false positives) who are entered into an intensive treatment regime is going to exacerbate existing problems. For example, patients suspected of sepsis are often over prescribed antibiotics and under-prescribed antiviral medications or the patients may receive inappropriate treatment for a period of time due to misdiagnosis.

Various attempts have been made to improve the precision and accuracy with which sepsis can be identified. For example, a range of diagnostic tests have been developed which identify specific biomarkers within bodily specimens gathered from patients, including those for specific subpopulations (e.g. those who recently underwent a surgical procedure), and those for detecting temporal changes in the biomarker. These approaches have largely been aimed at predicting sepsis as precisely as possible, as far in advance as possible, as well as separating sepsis from other conditions related to organ failure and systematic inflammation and for monitoring sepsis patients' conditions (e.g. to provide prognostic information and information for patient stratification). However, one disadvantage is that these require the procurement of bodily specimen(s) using invasive tests (e.g. blood test) and thus are unlikely to be performed outside of clinical, and typically hospital, settings. Further these tests can often take considerable time (typically several hours to days) to confirm the presence of the condition. As such their use is mostly limited to hospital settings, and prescribing such tests also requires experienced/vigilant clinicians. A further complicating factor is that during admission many presenting patients are initially seen by junior/less experienced clinicians who either fail to, or who require authorisation, to prescribe such tests. This further delays detection and treatment with consequent adverse outcomes for patients.

Another significant issue is that a very high proportion of sepsis cases begin outside of the hospital where access to the laboratory tests and monitoring required for SOFA/qSOFA is much more limited. Patients with infections outside of hospital settings may not seek treatment until the patients feel very sick and uncomfortable and the condition has progressed to its later moderate or severe stages where the symptoms are clearly perceivable (and more life threatening and costly to treat).

Accordingly, a range of computationally based prediction systems have been developed. Some of these systems incorporate expert knowledge. These systems, however, often include static reasoning/rules. Like the current rule-based systems already employed in hospitals (e.g. SIRS, SOFA, qSOFA), these suffer from a lack of precision. More recently machine learning and Artificial Intelligence (AI) based systems have been proposed as such systems can learn to predict sepsis from a range of data sources. Whilst these methods show potential, they have suffered from a number of deficiencies which has limited their wide spread use.

In particular, many machine learning and AI systems rely on data or information that is typically only collected in critical/intensive care settings and thus are not sufficiently robust or suitable for use in other settings where sepsis development can be most prevalent (e.g. in the community or on hospital wards). Whilst some machine learning and AI systems have been designed to use more objective and easily obtained measurements, for example using non-invasive medical and consumer devices that allow for the measurement and tracking of vital signs outside of clinical environments, to date such systems have lacked the necessary precision to differentiate septic patients from non-septic patients in the general community.

Further machine learning and AI systems suffer from issues relating to the training data. Sepsis manifests in patients in a wide number of ways and thus machine learning and AI systems must be able to robustly predict sepsis in many different population groups. However, many systems have only been trained on heterogeneous populations (e.g. patients in the ICU) and there are recent suggestions that this heterogeneity can be limiting with respect to precision depending on the population in which predictions are being made. Recently some investigators studying other health conditions (ICU mortality and diabetes) have proposed using similarity measures to identify like patients, and have shown that models trained on smaller groups of like patients perform better than models trained on full datasets with heterogeneous populations. However, this research is in its early stages and there remain many uncertainties in extending this work to other health conditions, such as the size of the like population and the choice of similarity measure, particularly in a complex disease state such as sepsis. Further this work used static datasets, and it is unclear whether such performance would be achieved in a fully automated live system where there are repeated and dynamic/updating patient measures.

Further the populations that are used to evaluate machine learning models can be manipulated to skew the standard performance metrics found in the literature and thus misrepresent the practical use of proposed models. For example, the same prediction model can produce quite different prediction outcomes based on what combination of a feature set is used, as well whether a heterogenous or homogenous study population is used for training and testing. Deployment of non-robust models can lead to high false positive rates (low positive predictive value), which has been shown to adversely influence adoption and usefulness of such technologies.

Sepsis is one of the most deadly and costly medical conditions and there is a need to develop precise and robust early detection methods and systems for sepsis for patients in the general community and general hospital wards, or to at least provide a useful alternative to existing methods and systems. Such methods and systems would enable rapid identification and timely intervention thus leading to improved overall clinical outcomes, reduced costs, and more importantly enhanced patient survival.

### SUMMARY OF THE INVENTION

According to a first aspect, there is provided a computational method for detection of infection and sepsis, the method comprising:
storing patient health data in a data store for a plurality of patients, the patient health data for a patient comprising a plurality of data items comprising a plurality of clinical data items obtained from one or more clinical data sources, a plurality of patient measurement data obtained from one or more wearable, home, and community-based biomedical sensors, and a plurality of symptoms obtained from the patient;
generating and storing a plurality of sepsis prediction models and a general population sepsis prediction model each trained using the stored patient health data wherein the general population sepsis prediction model is generated by training a sepsis prediction model on a general population of patients drawn from the plurality of patients and generating each of the plurality of sepsis prediction models comprises:
   identifying a training cohort of similar patients according to a patient similarity measure wherein each patient similarity measure is determined using a different combination of data items in the patient health data, one or more similarity functions and/or one or more similarity criterion; and
   training a sepsis prediction model using the training cohort of similar patients;
obtaining patient health data for a monitored patient, the patient health data comprising a plurality of clinical data items obtained from one or more clinical data sources, a plurality of patient measurement data obtained from one or more wearable, home, and community based biomedical sensors, and a plurality of symptoms obtained from the patient;
selecting a sepsis prediction model from the plurality of sepsis prediction models for monitoring the monitored patient, where the sepsis prediction model is selected by identifying the sepsis prediction model with the training cohort most similar to the monitored patient, and if no similar training cohort can be identified then selecting the general population sepsis prediction model;
using the selected sepsis prediction model to monitor the monitored patient to detect infection and sepsis events, and generating electronic alerts if an infection and sepsis event is detected;
repeating the selecting step in response to a change in the patient health data of the monitored patient over time; and
repeating the generating and storing step in response to one or more confirmations of detected infection and sepsis events.

In one form, the one or more clinical data sources may comprise electronic medical records and a clinician user interface configured to receive clinical notes from a clinician.

In one form, the one or more of the plurality of patient measurement data may be obtained from the monitored patient comprises repeated measurements of one or more vital signs, with each measurement having an associated time.

In one form, the one or more personal, home, and community based biomedical sensors comprise one or more wearable sensors and vital sign sensors.

In one form, the one or more of the plurality of patient symptoms may be obtained from the monitored patient are obtained and entered using a patient user interface executing on a mobile computing apparatus.

In one form, each sepsis prediction model may be a machine learning classifier which is configured to monitor updates to patient health data for the monitored patient and generate an alert if an infection and sepsis event is detected.

In one form, identifying the sepsis prediction model with the training cohort most similar to the monitored patient may comprise:
filtering the plurality of sepsis prediction models to identify a set of similar models based on one or more current symptoms and one or more current vital signs of the monitored patients; and
selecting a model from the set of similar models based on the model in the set of similar models in which the training cohort of patients have the most similar set of medical conditions to the monitored patient.

In a further form,
filtering the plurality of sepsis prediction models may be performed by calculating a similarity score for each model which is a weighted sum of the of the similarity between one or more current symptoms and one or more current vital sign measurements of the monitored patient, and the corresponding one or more symptoms and one or more vital sign measurements of patients in the training cohort of the respective model; and
selecting a model from the set of similar models may be performed by calculating, for each model in the set of similar scores, a total similarity score by, for one or more medical conditions, determining a similarity score between the medical condition of the monitored patient and corresponding medical condition for each patient in the training cohort of the respective model and multiplying the similarity score by a weight for the medical condition, and then summing each of the weighted similarity scores to obtain the total similarity score; and selecting the model with the highest total similarity score.

In one form, storing a plurality of trigger conditions and repeating the selecting step may be performed in response to an update in patient health data satisfying one or more of the trigger conditions.

In one form, the electronic alert may comprise an alert to a clinician via a clinician user interface, and wherein the clinician user interface is configured to allow the clinician to confirm the validity of the infection and sepsis event, and one or more confirmations are used to trigger repeating the generating and storing step.

According to a second aspect, there is provided a computational apparatus configured for the detection of infection and sepsis in a monitored patient, the apparatus comprising:
one or more processors;
one or more memories operatively associated with the one or more processors;
a data store configured to store patient health data for a plurality of patients, the patient health data for a patient comprising a plurality of data items comprising a plurality of clinical data items obtained from one or more clinical data sources, a plurality of patient measurement data obtained from one or more wearable, home, and community based biomedical sensors, and a plurality of symptoms obtained from the patient via a patient user interface;
wherein the one or more memories comprise instructions to configure the one or more processors to:
generate and store in a model store a plurality of sepsis prediction models and a general population sepsis prediction model each trained using the stored patient health data obtained from the data store, wherein the general population sepsis prediction model is generated by training a sepsis prediction model on a general population of patients drawn from the plurality of patients and generating each of the plurality of sepsis prediction models comprises:
   identifying a training cohort of similar patients according to a patient similarity measure wherein each patient similarity measure is determined using a different combination of data items in the patient health data, one or more similarity functions and/or one or more similarity criterion; and
   training a sepsis prediction model using the training cohort of similar patients;
obtain patient health data for a monitored patient, the patient health data comprising a plurality of clinical data items obtained from one or more clinical data sources, a plurality of patient measurement data obtained from one or more wearable, home, and community based biomedical sensors, and a plurality of symptoms obtained from the patient;
select a sepsis prediction model from the plurality of sepsis prediction models for monitoring the monitored patient, where the sepsis prediction model is selected by identifying the sepsis prediction model with the training cohort most similar to the monitored patient, and if no similar training cohort can be identified then selecting the general population sepsis prediction model;
use the selected sepsis prediction model to monitor the monitored patient to detect infection and sepsis events, and generating electronic alerts if an infection and sepsis event is detected;
repeat the selecting step in response to a change in the patient health data of the monitored patient over time; and
repeat the generating and storing step in response to one or more confirmations of detected infection and sepsis events.

In one form, the one or more memories may further comprise instructions to further configure the one or more processors to provide a clinician user interface wherein the one or more clinical data sources comprises electronic medical records and the clinician user interface is configured to receive clinical notes from a clinician.

In one form, the one or more of the plurality of patient measurement data may be obtained from the monitored patient comprises repeated measurements of one or more vital signs, with each measurement having an associated time.

In one form, the one or more personal, home, and community based biomedical sensors comprise one or more wearable sensors and vital sign sensors.

In one form, the one or more of the plurality of patient symptoms may be obtained from the monitored patient are obtained and entered using a patient user interface executing on a mobile computing apparatus.

In one form, each sepsis prediction model may be a machine learning classifier which is configured to monitor updates to patient health data for the monitored patient and generate an alert if an infection and sepsis event is detected.

In one form, identifying the sepsis prediction model with the training cohort most similar to the monitored patient may comprise:
filtering the plurality of sepsis prediction models to identify a set of similar models based on one or more current symptoms and one or more current vital signs of the monitored patients; and
selecting a model from the set of similar models based on the model in the set of similar models in which the training cohort of patients have the most similar set of medical conditions to the monitored patient.

In a further form,
filtering the plurality of sepsis prediction models may be performed by calculating a similarity score for each model which is a weighted sum of the of the similarity between one or more current symptoms and one or more current vital sign measurements of the monitored patient, and the corresponding one or more symptoms and one or more vital sign measurements of patients in the training cohort of the respective model; and
selecting a model from the set of similar models may be performed by calculating, for each model in the set of similar scores, a total similarity score by, for one or more medical conditions, determining a similarity score between the medical condition of the monitored patient and corresponding medical condition for each patient in the training cohort of the respective model and multiplying the similarity score by a weight for the medical condition, and then summing each of the weighted similarity scores to obtain the total similarity score; and selecting the model with the highest total similarity score.

In one form, the one or more memories may be configured to store a plurality of trigger conditions and repeating the selecting step is performed in response to an update in patient health data satisfying one or more of the trigger conditions.

In one form, the one or more memories may comprise instructions to further configure the one or more processors to provide a clinician user interface, wherein the electronic alert comprises an alert sent to a clinician via the clinician user interface, and the clinician user interface is configured to allow the clinician to confirm the validity of the infection and sepsis event, and one or more confirmations are used to trigger repeating the generating and storing step.

### BRIEF DESCRIPTION OF DRAWINGS

Embodiments of the present disclosure will be discussed with reference to the accompanying drawings wherein:

FIG. 1 is a flowchart of method for the detection of infection and sepsis according to an embodiment;

FIG. 2 is a schematic diagram of a system for the detection of infection and sepsis according to an embodiment;

FIG. 3 is a schematic diagram illustrating a high-level process workflow showing the exchange of information between various components of a system for the detection of infection and sepsis according to an embodiment;

FIG. 4 is a schematic diagram of an infection and sepsis forecaster module which is used to select the most appropriate model to classify a patient according to an embodiment; and

FIG. 5 is a data flow diagram of the classifier component within the infection and sepsis forecaster according to an embodiment;

In the following description, like reference characters designate like or corresponding parts throughout the figures.

### DESCRIPTION OF THE INVENTION

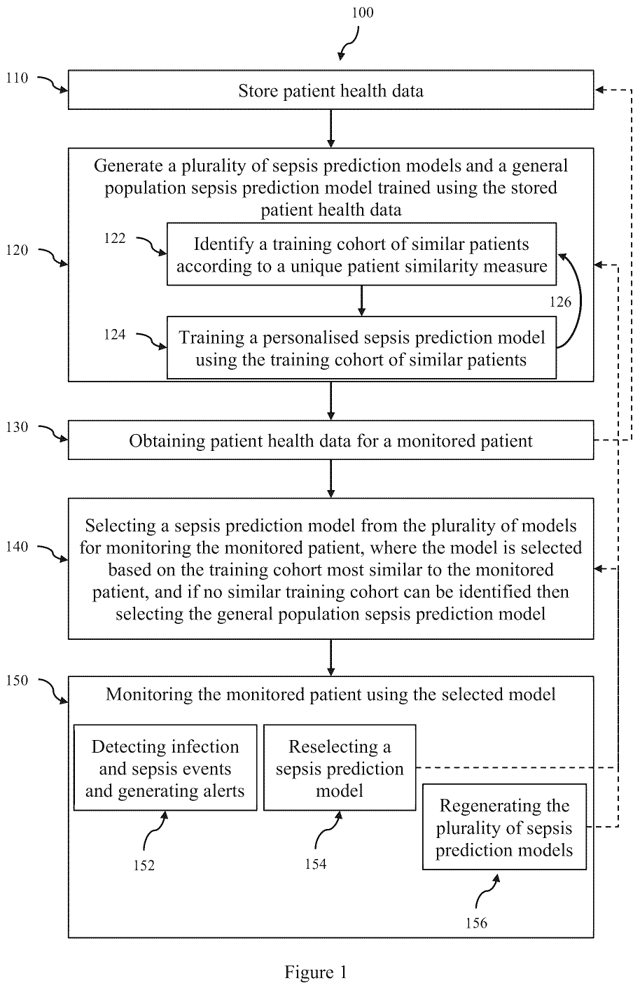

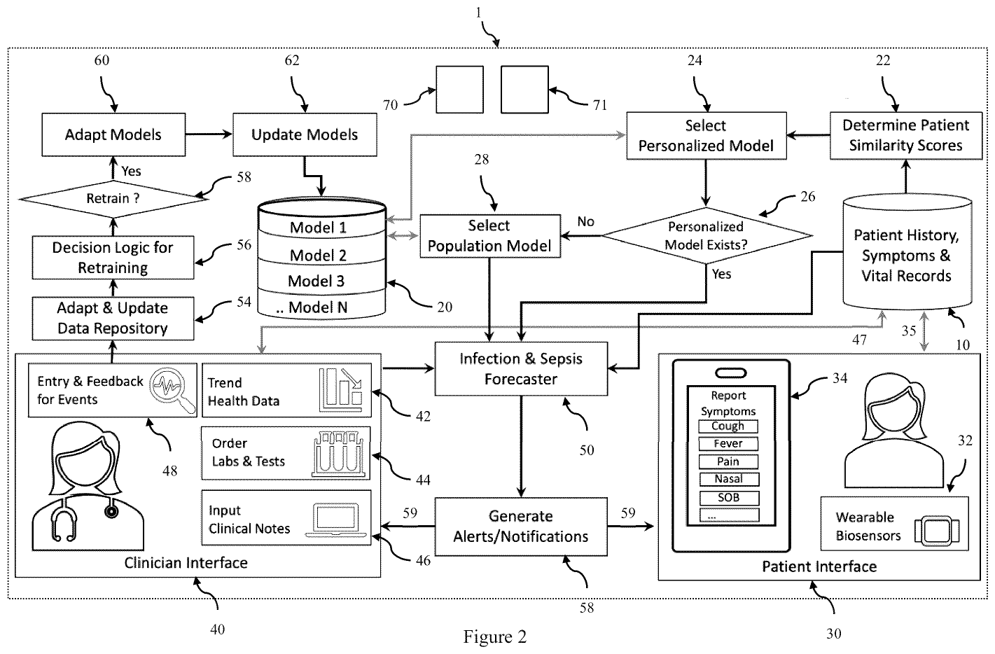

Referring now to FIGS. 1 and 2, there is shown a flowchart of method 100, and schematic diagram of a system 1, for the detection of infection and sepsis according to an embodiment, and embodiments of the system 1 may be configured to implement the method 100. Embodiments may allow for the personalized prediction of infection and sepsis in a patient to enable more precise and robust early detection and thus improve patient outcomes. Embodiments of the system are configured to store patient health data 110 for a plurality of patients. The patient health data may be stored in a data store 10, which may be a database or a multiple connected databases stored on local, networked, or cloud based storage devices. The patient health data for a patient comprises a plurality of data items comprising a plurality of clinical data items obtained from one or more clinical data sources, a plurality of patient measurement data obtained from one or more wearable, home and community based biomedical sensors such as wearable and vital sign sensors 34, and a plurality of symptoms obtained from the patient, for example via a patient user interface 30 executing on a mobile computing device 34 used by the patient. The plurality of patients may comprise both historical patients and monitored patients. Historical patients are patients for which historical patient health data may be available and may include previously monitored patients. In the context of the following description, the focus will be on a monitored patient and how the system is used to monitor and detect infection and sepsis using a personalized model in this monitored patient. It will be understood, however, that embodiments may be used to simultaneously monitor many patients, each with a personalized monitoring model.

The system and method are configured to generate a plurality of sepsis prediction models and a general population sepsis prediction model each trained using the stored patient health data 120. These models may be machine learning or AI-based models, such as classifier models, and may be stored in a model store 20, such as a database or file store that electronically stores the relevant model parameters and configuration (for example by exporting a trained model) to allow later use of the stored model. Generating each of the plurality of sepsis prediction models may comprise identifying a training cohort of similar patients according to a patient similarity measure 122 and then training a sepsis prediction model using the training cohort of similar patients 124. Each time this is performed a different similarity measure is used based on a different combination of data items in the patient health data, one or more similarity functions and/or one or more similarity criterion to generate a different training cohort, and thus a different model. The data items could be different symptoms, measured vital signs and disease conditions. Each similarity measure is thus a distinct measure to enable generation of a distinct model, and through repeating this process we can generate a plurality of distinct (or unique) models. The patient similarity measure could be determined using one or more similarity scores, similarity metrics, similarity functions, or similarity criterion (or criteria), including various combinations of these, applied to various combinations of data items such as symptoms, measured vital signs and disease conditions. The patient similarity measure is then used to identify similar patients. For example, a first similarity measure could be determined using a scoring function calculated using a set of 10 data items, whilst another similarity measure could be calculated using a set of 20 data items. Note that two models could use the same cohort of training patients with each model using a different combination of the data items to train/build the model. In some embodiments, a similarity function may be used to generate a similarity score, and the score may be used directly as the similarity measure with similar patients selected based on having a score exceeding a threshold. In this embodiment the threshold is a similarity criterion, and thus different groups of similar patients could be identified using the same scoring function but using different thresholds (i.e. different similarity criterion). In another embodiment, similarity measures may be calculated for all patients, and the N (e.g. 500, 1000, 5000, 10000) patients with the highest similarity scores selected. In some embodiments a similarity score may be transformed or combined to obtain a numeric similarity measure. e.g. to convert the score to a similarity probability or to normalize the score to a predefined range such as [0,1]. In some embodiment a single similarity score may be calculated using a specific similarity function, whilst in other embodiment several similarity scores could be calculated each using different similarity function with the similarity scores added. The different scores could be combined using simple addition or some weighted combination (including linear and non-linear combinations). In some embodiments, similarity criterion (or criteria) could be used to require or weight specific conditions (e.g. diabetes), and this could be applied as a multiplier to a similarity score, or the multiplier could be integrated into a similarity function used to calculate a score. The weighting factor could be a binary (0,1) value to force the presence of a specific criterion, or each similarity criterion could have a numeric value over some range such as [0,1] that could be either predefined for a specific use-case configuration or autonomously learnt from the dataset and continuously adapted by the dataset. In some embodiments, the similarity measure may be a class rather than a numeric value. e.g. a patient may be directly assigned a class, for example by using a trained similarity classifier model, or a similarity score may be calculated and assigned to a similarity class. The set of similarity classes could be binary (similar, not similar) or multi-class (highly similar, similar, dissimilar, or highly dissimilar). Similar patients could then be identified based on their class, for example only similar patients, only highly similar patients, or highly similar or similar patients, or even not highly dissimilar patients. A random or partially random process could be used to create the patient similarity measure. For example, the number of data items used to determine a patient similarity measure (or score) could be varied, leading to some general similarities measures as well as narrow similarity measures. In some embodiments, the data items are randomly selected from all data items, and in other embodiments, a patient similarity criterion (or criteria) is determined by randomly selecting from different subsets of the patient health data, such as at least one clinical data item, at least one patient measurement (e.g. physiological data), and at least one symptom. Further a given subset may be further divided into subtypes (or levels). For example, clinical data items could be further divided into demographic/descriptive data of the patient (age, weight, sex, smoking status, etc.), pre-existing medical conditions (diabetes, heart disease, allergies, etc), and clinical observations/notes. Similarity between patients may be assessed based on correlation measures, scoring systems, distance measures, etc. When generating a specific combination of data items, similarity functions, and/or similarity criterion/criteria used to generate a similarity measure/similar group of patients, a check could be performed to ensure the current combination is sufficiently different from another set (for example at least 3 different data items selected). Similarly after multiple similar patient groups have been identified using different similarity measures, this set could be filtered to exclude a patient group too similar to another patient group to ensure a diversity of similar patient groups, and thus a diversity of sepsis models. The models may be trained using all available data for patients (for example using deep learning training methods), or using specific data items, which may be determined based on how the similar patients were identified, for example the same set of data items used to calculate similarity. A general population sepsis prediction model is also generated by training a sepsis prediction model on a general population of patients drawn from the plurality of patients. This may be all the patients in the data store or a random or representative sample. Similarity measures could be calculated between patients in the samples to ensure the sample is reflective of a general population (for example by requiring the average similarity to be low). That is, models are trained on a range of homogenous sub-populations with similar health data as well as a model based on a general heterogenous population.

As noted above, the system may be used to monitor patients in order to detect infection and predict sepsis development well in advance. For the following the monitoring of a specific patient will be considered. The system is configured to obtain patient health data for the monitored patient 140. As outlined above the patient health data may comprise a plurality of clinical data obtained from one or more clinical data sources, a plurality of patient measurement data obtained from one or more wearable, home, and community based biomedical sensors such as wearable sensors and/or vital sign sensors (including non-invasive and invasive vital sign sensors), and a plurality of symptoms obtained from the patient. When the patient is first monitored, patient health data may be captured or imported from electronic health records and clinical record systems, or access may be provided to the electronic health records or systems. Moving forward, the system can continuously monitor the patient collecting regular or ad-hoc patient health data from wearable and home/clinic based vital sign sensors, as well as symptoms. Updates may also be obtained from clinical data sources, such as laboratory test results, treatments and clinician notes. The system is configured to select a sepsis prediction model from the plurality of sepsis prediction models for monitoring the monitored patient. This is performed by identifying the sepsis prediction model with the training cohort most similar to the monitored patient (i.e. “like patients”), and if no similar training cohort can be identified then selecting the general population sepsis prediction model 140.

The selected sepsis prediction model is then used to monitor the monitored patient to detect infection and sepsis events 150, for example by processing new/updates to patient health data. This may be used to generate electronic alerts if an infection and sepsis event is detected 152. The system may also repeat the step of selecting the most similar sepsis prediction model 140 in response to a change in the patient health data of the monitored patient over time 154. This allows the system to keep using the most similar (and arguably relevant) patient cohort as the patient's measurements and symptoms change, for example as the monitored patient begins to show signs of an infection or sepsis. Additionally, the system may be triggered to repeat the step 120 of generating and storing a plurality of sepsis prediction models 156. This may be in response to one or more confirmations of detected infection and sepsis events, once a threshold time has passed, or once a threshold additional number of patients have been enrolled into the system. This allows the system to continuously adapt and update as new information and patients is received.

Embodiments of the system are further illustrated in FIGS. 2 to 5. The system may be implemented on a computational apparatus comprising one or more processors 70 and one or more memories 71. The computational apparatus may comprise one or more servers, including one or more cloud-based servers or other distributed computing environment. The memory may store software or instructions to implement the method including controlling the training of models, data collection and interfaces, and generation of alerts. To obtain patient health data from a monitored patient, a patient interface 30 is provided to enable collection of patient measurement data obtained from wearable and/or vital sign sensors 32, and symptoms 34 for example by a patient user interface executing on a computing device used by the patient, such as a smart phone, tablet, laptop or desktop computer. The patient user interface may be provided as an application (app) installed on a smart phone or tablet.

The patient is intermittently or continuously monitored by one or more home and community based biomedical sensors. This may include one or more wearable devices that measure the patient's physical activity, the patient's physiological vital signs, along with other parameters related to patient health. The physical activity measures may include, but not limited to, body acceleration, steps, posture, activity intensity, ambulation, gait and associated durations. The physiological measurements may include, but not limited to, heart rate (or pulse rate), heart rate variability, respiratory rate, temperature, and derived parameters. Additional parameters may include, but are not limited to, those related to edema and swelling such as weight and extremity size. Data may also be collected from non-invasive vital sign sensors provided in a home, community health clinic or general practitioner office (i.e. non-hospital), such as blood pressure monitors and heart rate monitors. Data could also be collected from personal/home invasive/semi-invasive or sample-based sensors such as subcutaneous implantable sensors such as those used for blood glucose monitoring. These can be generally classed as home and community based biomedical sensors (as distinct from hospital-based monitoring equipment). A patient interface is provided to connect to and download data from the sensors. This may be directly from the sensors, for example via an app running on a local smartphone or computing device, or from other storage sources, such as cloud storage sources. A user interface 34 is also provided to allow collection of patient symptoms. This may be an application installed on the patient's smartphone or tablet (or other computing device) and allows the patient to enter from time-to-time the commonly experienced symptoms associated with his/her health such as cough, fever, pain, nasal congestion, shortness of breath and other signs. This patient health data is sent or uploaded 35 to a patient data store 10. This may be secure data storages including, but not limited to, dedicated hard disks on servers or cloud storage services. This may be sent in real time, periodically, or in batches. The monitoring data may be continuous or intermittent, and may comprise repeated measurements of one or more vital signs, with each measurement having an associated time.

A clinician interface 40 is also provided to interface with the one or more clinical data sources such as electronic medical records and a clinician user interface. The clinician user interface also allows the clinician (including doctors, surgeons, medical specialists and other health care professionals and service providers) to access or visualize the patient's health data and trends from a set of dashboards or summary pages or graphic illustrations on mobile application or website portals 42. The clinician is able to input clinical interpretations and observational notes into the system via appropriate user interfaces including submitting text summaries of patient status and interactions. Laboratory (lab) test reports, images and documents may also be viewed or imported, uploaded or access granted 44. As will be discussed below, the clinician is also able to review push notifications of alerts on sepsis detector 50 outputs and wearable sensor notifications and provide clinical feedback regarding whether the generated alerts are true positives or potentially false alarms 48. All the clinician inputs and notes are transferred 47 to the secured cloud/data storage of the system 10. The clinician interface 40 may also be configured to access electronic medical records stored by hospitals, clinics, or other health providers which contain the patient's demographic characteristic profile (i.e. patient metadata) and clinical history, including outcomes of infections, treatments and hospitalizations. Such outcomes in relation to infection and sepsis events can be used to train the sepsis prediction models. Patient metadata such as demographic information, general health characteristics and pre-existing conditions may also be entered via the patient user interface or clinician user interface.

The data store 10 used to store patient health data also provides the data to the infection and sepsis forecaster 50 which is used to monitor the patient using a personalized sepsis prediction model and detect infection and sepsis events. The data transfer between the data storage 10 and the clinician interface 40 and patient interface 30 are bidirectional, in which the data can be retrieved and also pushed with new data or update the existing data content.

As outlined above, the system determines patient similarity measures, such as similarity scores 22 based on the monitored patient's health data such as the patient's history, symptoms and continuously updating vital sign and measurement records to each of the training cohorts, and then uses these scores to select a personalized sepsis prediction model 24 from the set of pre-trained sepsis predictive models stored in the model store 20. As noted above each of these models are pretrained for a set of similar patients (“like patients”), referred to as the training cohort for the respective model, with similarity measures calculated from their health characteristics including history, comorbid conditions, symptoms, physiological measurements and laboratory values. As discussed above, the process to compute the similarity measure could use any or combinations of data items (or encodings) of the patient health data, similarity functions/metrics (which may generate similarity scores), and/or similarity criterion/criteria. There is also no restriction in the methods by which the similarity of various parameters is assessed or how these similarity functions/scores/criterion are combined and or applied to obtain a patient similarity measure. If a matching personalized sepsis prediction model exists 26, the predictive model parameters are input to the infection and sepsis forecaster 50, which extracts the relevant patient's history, symptoms and vital records from the data storage, and the processed health data trends, laboratory test results, clinician notes from clinician interface tools as well, as shown in FIG. 2. As noted above, the model store 20 also contains a model pretrained for the general population. In the case that the input patient characteristics are a very unique corner cases, or found to be less similar to the existing previous patient pool, or if a personalized predictive model does not exist to match for the input patient characteristics, the general population based model is selected 28 and the respective model parameters are input to the infection and sepsis forecaster 50.

The infection and sepsis forecaster 50 uses the selected sepsis prediction model to monitor the incoming patient data. The sepsis prediction model may be any or a combination of a rule-based infection and or sepsis event detector, binary or multi-class classifier or a multivariate regressor assessing the risk for infection and sepsis events based on the monitoring data. The sepsis prediction model is configured to analyse incoming patient health data and generate an output indicating the risk of one or more infection and sepsis events. This may be a binary outcome, likelihood score or a probability measure. Determination of a positive event or a class or a risk associated with infection and sepsis leads to the generation of alerts and notifications 58. In one embodiment each sepsis prediction model is a machine learning classifier which is configured to monitored updates to patient health data for the monitored patient and generate an alert if an infection and sepsis event is detected.

Alerts 59 may be sent to the patient or their caregiver via the patient user interface 34, for example to alert them to a potentially serious infection or deterioration event. Alerts 59 may also be sent to the clinician interface to notify the clinician, health care provider and associated parties and displayed in the clinician user interface, for example on a mobile application and or web portal. Additional data such as health trend data may be included with the alert. The clinician can review the generated positive alerts 59, the corresponding health trend data, and can verify the validity of the generated alerts and provide a feedback in annotating the infection and sepsis events to be true positives or false positives 48. In case of a new clinical event, the clinician interface allows the clinician to make entries of clinical events including severe adverse events and changes in medications. The clinician's feedback for the generated infection and sepsis events or the new entries of clinical events are pushed and updated as the corresponding reference data for the given patient's health information, measurements and symptoms 54.

Upon the adapting and updating of the data repository 54, a decision for retraining of the infection and sepsis prediction model is obtained either automatically at desired periodic time intervals or with manual confirmation input using clinical interface tool 56. The automatic decision logic for retraining may be enabled or disabled depending upon desired preset criterion (or criteria). In one embodiment, if the feedback entries for generated positive infection and sepsis events and or the new entries of qualifying clinical events exceeds a preset threshold, then decision logic is enabled for retraining 58 that results in adaptation or regeneration of the infection models sepsis prediction model for the given data repository containing patent information, continuous and or discrete patient measurements, and episodic symptoms and reference events 60. After retraining the models 156, the updated models 62 are stored in the model store 20, for example replacing the currently stored models.

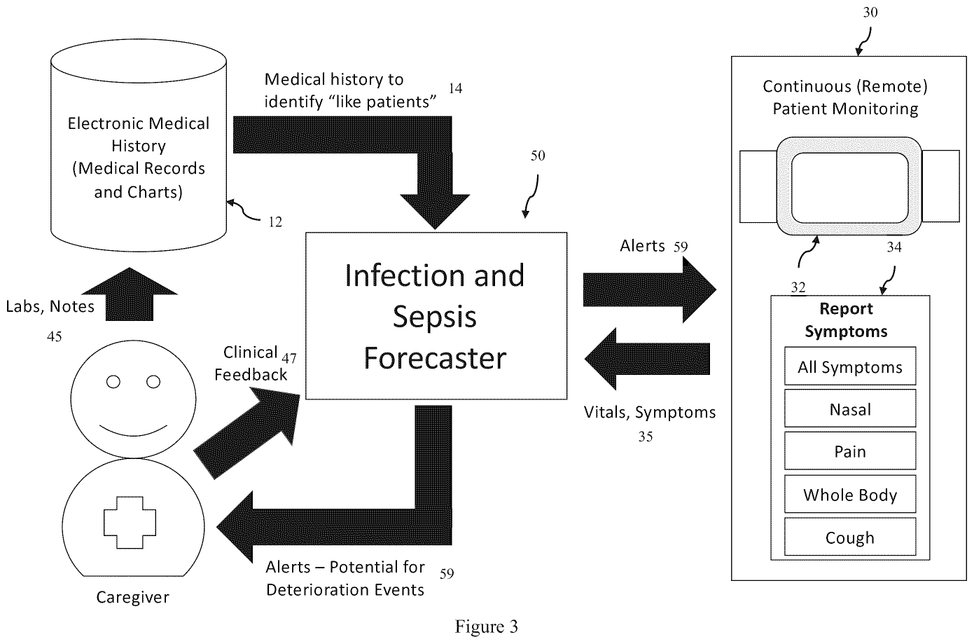

This is further illustrated in FIG. 3, which is FIG. 3 is a schematic diagram illustrating a high-level process workflow showing the exchange of information between various components of a system in the detection of infection and sepsis according to an embodiment.

As shown on the right-hand side of FIG. 3, the patient interface 30 is configured to continuously monitor the patient by one or more wearable device measuring common vital signs 32. The patient may also use a companion application 34 on a smartphone or tablet and voluntarily reports symptoms, questionnaire inputs and outcome measurements. Requests, reminders or notifications for such information could be also be pushed to the patient's social media feed or social media apps. The self-reports as well as the vitals serve as some of the inputs into the infection and sepsis forecaster 50, which is responsible for alerting the patient and/or patient's caregivers to potential serious infection and deterioration events.

As shown on the left-hand side of FIG. 3, the patient's electronic health records 12 also serve as input to this patient monitoring system. As new information is added by an attending caregiver 47, and laboratory tests and notes 45 are added to the medical records 12, these updates can be pushed to the infection and sepsis forecaster 50. Alerts 59 can allow for a patient and caregivers to make proactive decisions to seek testing and treatment for infections early so that they do not progress. Feedback from patients and caregivers based on these alerts can close the loop and allow for the infection and sepsis forecaster to continually learn and improve as new patients are incorporated into existing sepsis classifiers.

Vital signs have shown predictive power for the purposes of identifying and predicting sepsis onset, and it is becoming increasingly more common and easier for individuals to incorporate devices into their daily lives that can measure these signals continuously. A single wearable device, for example, can incorporate sensors that measure an array of parameters related to cardiovascular health, respiration, and temperature regulation. These devices can continuously send timecoded (time stamped) readings of these signals to the infection and sepsis forecaster 50. In one embodiment, the infection and sepsis forecaster is a machine learning classifier model. The sepsis classifier applies the trained model to these data streams that has learned parameters (for example, thresholds for gradient boosted decision trees) from the data of previous patients. The classifier will be able to use these set parameters (thresholds) to make an assessment about this patient in near real time to decide if the patient or caregiver needs to be alerted about potential impending deterioration events. Rather than using a set parameter model, the system continuously identifies and selects the most appropriate trained prediction model based on identifying the most similar training cohort based on the current measurements and symptoms. This provides a dynamic and personalized prediction system. Further, as additional data and patients are added, the system dynamically adapts by regenerating the plurality of models, and selecting the most similar model for the regenerated models (and training cohort of similar patients).

An embodiment of how the most similar model, or rather the model based on the most similar training cohort, is selected, as well as reselection/updating and regeneration/adaption processes will now be described.

Sepsis prediction problems and the outcomes of this condition differ depending on the type and severity of the infection that the patient is battling and the different types and stages of sepsis. If a patient is battling a respiratory infection rather than a urinary tract infection (UTI), it makes sense that the classifier to identify whether or not the patient will become septic should use a model that learned its parameters from patients who were battling similar infection conditions.

In this embodiment, the patient can report symptoms that are good indicators that the patient is battling an infection of some kind (for example, fever) as these may be the only types of symptoms that are present; however, additionally, the patient may report symptoms that are indicative of certain type of infection (for example, labored breathing for respiratory infections). In the example, the patient can report a symptom along with its severity (via user interface 34). The way in which patient self-report and symptom severity are assessed here is exemplary and may be varied in other embodiments. A large number of symptoms can be enumerated for the patient as well as the severity. For example, a cough can be dry or wet/productive, a fever can be slight (99-100° F.), high (100-102° F.), or dangerous (>103° F.), etc. Symptoms could be encoded alternatively, binary, present or not present, on a continuous scale of severity, etc.). These can then be used to identify patients with like symptoms, vitals, and thus likely to have had a similar disease course to the current patient, and thus enable selection of the model trained on the most similar patient cohort.

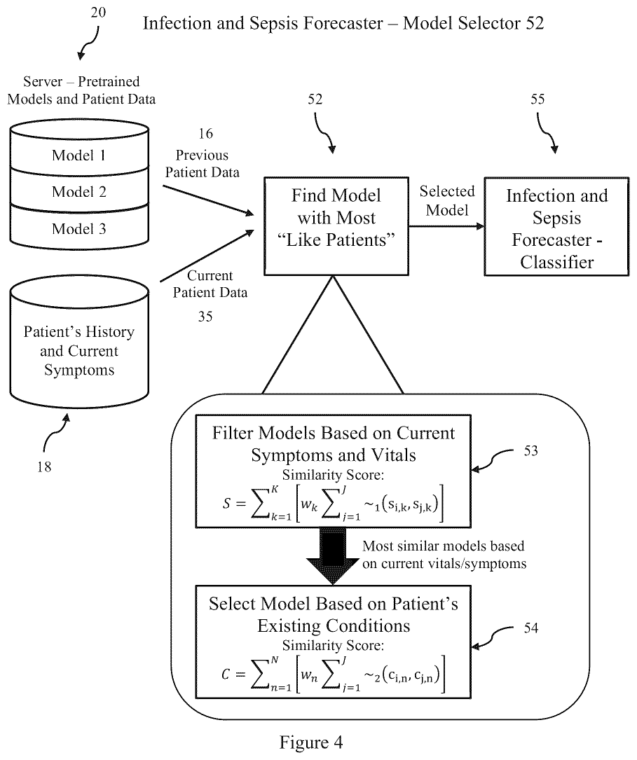

This is further illustrated in FIG. 4 which is a schematic diagram of an infection and sepsis forecaster module which is used to select the most appropriate model to classify a patient according to an embodiment. As shown in FIG. 4, within the infection and sepsis forecaster 50, there is a model selector component 52 that uses the current patient's vitals, symptom 35 and medical history data 16 to select the sepsis prediction model 52 which in this embodiment is a machine learning-based classifier 55. The model selection 52 is done by substituting one pre-trained model/classifier for another one (i.e. the parameters/thresholds that have been learned from one population are replaced by those learned from another (more similar) population). When the patient's symptoms are updated by the self-report application 34, or when the patient's electronic medical history is updated, the model/classifier that is used is updated or re-selected. This is to ensure the patient is being compared to the current most “like patients”.

In this embodiment, identifying the sepsis prediction model with the training cohort most similar to the monitored patient is a two-step process in which the plurality of sepsis prediction models is first filtered to identify a set of similar models based on one or more current symptoms and one or more current vital signs of the monitored patient at step 53. A model is then selected from the set of similar models based on the model in the set of similar models in which the training cohort of patients have the most similar set of medical conditions to the monitored patient at step 54.

The first step 53 is to ensure the model used to classify the current patient's data was constructed (the parameters/thresholds were set) using patients with similar vitals and symptoms. These two feature types are used in this similarity computation example, but additional information could be incorporated in other embodiments. As described above, sepsis development will likely depend on the type of infection the person is battling. Patients battling respiratory infections are going to have distinct symptoms, vitals and experiences compared to those who have a skin/tissue infection. Both may develop a fever but while one may have pain in the area of a cut/wound, the other may be short of breath or have wet cough from the outset of the illness.

The second step 54, in this example, is to ensure that the model selected to monitor the current patient has been constructed with patients who share a similar medical history to the current patient. If the current patient has chronic medical conditions that compromise their ability to fight off infection (e.g. a certain type of cancer being treated with chemotherapy, which compromises immune response), it is important to be compared with like patients. Similarly, if a patient has a condition that is going to meaningfully change their vital signs (e.g. chronic heart failure, hypertension, chronic obstructive pulmonary disease (COPD)), they can be compared to patients who had these systems similarly compromised.

In the embodiment shown in FIG. 4, a similarity score is computed for each pre-trained model. The way in which this similarity score is computed, including the way in which the features are encoded for the computation, are specific to the example and could be calculated differently in other embodiments.

In this embodiment, filtering the plurality of sepsis prediction models at first step 53 is performed using a vitals and symptom similarity score as the first filter. This is performed by calculating a similarity score for each model which is a weighted sum of the of the similarity between one or more current symptoms and one or more current vital sign measurements of the monitored patient, and the corresponding one or more symptoms and one or more vital sign measurements of patients in the training cohort of the respective model. That is a given model's vitals and symptom similarity score is a weighted sum of the similarity of the current patient's vitals and symptoms to the vitals and symptoms of the patients that were used to train the respective model. Each symptom value the current patient has reported (along with each lab result that has been added to his/her electronic record recently and each of the current vital signs) is compared against the values for that symptom reported by the previous patients (along with the lab test values reported by their caregivers and the vital signs that were recorded for them). The degree to which a symptom (lab values and vitals) corresponds to a previous patient's is determined by a unique function for that symptom (lab value, vital sign). The similarity values for that symptom (lab test, vital sign) are summed across all patients in the existing model. This summed value is multiplied by the weight of the symptom (lab test, vital sign). The weight for the particular symptom depends on its severity in the current patient and its uniqueness with respect to the type of infection. The final symptom similarity score for the model is the sum of these weighted individual symptom (lab test, vital sign) scores. When all of the models have had their symptom similarity scores computed, the models with scores above a set threshold are further considered. The models with similarity scores below this threshold are discarded and considered no further in this example.

In second step 54, a model is selected from the set of similar models by calculating, for each model in the set of similar scores, a total similarity score by, for one or more medical conditions, determining a similarity score between the medical condition of the monitored patient and corresponding medical condition for each patient in the training cohort of the respective model and multiplying the similarity score by a weight for the medical condition, and then summing each of the weighted similarity scores to obtain the total similarity score; and selecting the model with the highest total similarity score.

The second step 54 is thus very similar to the first step 53. Each of the remaining models has an existing condition similarity score computed. This similarity score is also a weighted sum. Each existing condition given in the current patient's electronic health records is compared to the existing conditions of the patients composing the model under consideration. The current patient's existing conditions are considered one at a time. If the previous patient had a similar existing condition (again similarity is determined by unique function for each existing condition), then the similarity score is added to a running sum. The summed score is multiplied by a weight for that existing condition. The weight depends on the conditions likelihood to compromise the patient's immune system or influence vital signals. These weighted existing conditions scores are summed for the final score. The model with the highest existing condition similarity score is selected to monitor the current patient.

To further illustrate this, an exemplary case will now be described which mirrors the higher-level example discussed above. Again, this is exemplary only and other variations may be used in other embodiment. Table 1 enumerates five different symptoms, along with their potential severity levels that could be reported by patients. It also has one vital sign related condition that could be noted from a remote monitoring device. Table 1 also gives a similarity function to assess each symptom's likeness. In this case, if the symptom values are either marked as the same (add 1) or not (add 0). Note that this is a small subset of the actual list of symptoms, vitals and severity levels that could be implemented. In this embodiment the case of a remotely and continuously monitored patient that has a respiratory infection will be considered. The patient might start with a dry cough, a mild fever (100° F.) and mild nausea. If the patient reports these, the system will adapt its classifier and monitor the patient. The symptom similarity score for each precompiled model would be a weighted sum of these three symptoms according to Equation 1. Each of the symptoms is considered in turn, and each patient in the precompiled model is considered in turn. If a previous patient has the same symptom severity value as the current patient, then one is added toward the similarity score. If they do not have the same value, then zero is added. The equation shown in FIG. 4 reduces to Equation 1 based on the values and definitions given in Table 1. As noted above, the weights are determined by the severity of the symptom and how unique it is when differentiating infections.

$$S = \{ \begin{matrix}1 * \# of patients with mild nausea + \\ 1 * \# of patients with dry cough + \\ 1 * \# of patients with low grade fever\end{matrix} \qquad Equation 1$$

These symptoms are common to any infection and reasonably mild, and so the weights are low. Models trained on more heterogenous populations will be selected. Let's say, however, that the fever worsens to a high grade fever (102° F.) and the patient begins to experience mild shortness of breath. The similarity function for “like patients” would change to Equation 2:

$$S = \{ \begin{matrix}1 * \# of patients with mild nausea + \\ 1 * \# of patients with dry cough + \\ 3 * \# of patients with high grade fever + \\ 3 * \# of patients with mild shortness of breath\end{matrix} \qquad Equation 2$$

The model selected is now going to include more patients with higher fevers and more patients with shortness of breath (similar to the current patient). This will presumably include more patients battling respiratory infections. The models with similarity scores above a set threshold are further considered in step two of the selection process.

TABLE 1
Five different symptoms, along with their potential severity levels that could be reported by patients, with
one vital sign related condition that could be obtained from a vital sign monitoring device, along with
associated weights and the similarity function used to compare the current patient to previous patients:

| Symptom | Values | wₖ | ~₁(s_{i,k}, s_{j,k}) |
|---|---|---|---|
| Shortness of Breath | None Very Mild | 0 3 | ${\sim}_{1} ( {s}_{i , k} , {s}_{j , k} ) = \{ \begin{matrix}1 , & if {s}_{i , k} = {s}_{j , k} \\ 0 , & otherwise\end{matrix}$ |
| | More Severe | 5 | If the precompiled model’s patient had the same |
| Nausea | None | 0 | symptom value as the current patient the value is |
| | Mild | 1 | 1; otherwise, the value is 0. |
| | Moderate | 3 | The more patients in the precompiled model with |
| | Vomiting | 5 | the same symptom as the current patient the |
| Cough | None | 0 | higher the symptom similarity score will be. |
| | Dry | 1 | |
| | Wet/Productive | 3 | |
| Fever | <100° F. (37.7° C.) | 0 | |
| | Low Grade: | 1 | |
| | 100° F.-101° F. | | |
| | (37.7° C.-38.3° C.) | | |
| | High Grade: | 5 | |
| | >101° F. (38.3° C.) | | |
| Pain - LLE | None | 0 | |
| | Mild | 3 | |
| | Moderate/Severe | 5 | |
| Tachycardia | HR <= 100 BPM | 0 | |
| | HR is >100 BPM | 3 | |
| [Other symptoms] | . . . | . . . | |

Table 2 summarizes some different chronic conditions that the patient might have, their weight, and the function used to compare the current patient to previous patients. This step is very similar to the previous step. If it is assumed that the patient only has chronic heart failure, then the model selected to monitor the patient will be one with the most chronic heart failure patients:

C=1*#of patients with chronic heart failure Equation 3

If the patient were to have chronic heart failure and hypertension (HTN), then the model selected would be biased toward patients with both of these conditions. Note that patients with both will add to the similarity score twice.

C=1*#of patients with chronic heart failure+1*#of patients with HTN Equation 4

The model with the highest existing condition similarity would then be selected and used to monitor the patient.

TABLE 2
Summary of some different chronic conditions that a patient might have, their weight, and the function
used to compare the current patient to previous patients:

| Symptom | wₖ | ~₂(c_{i,n}, c_{j,n}) |
|---|---|---|
| Breast Cancer Chronic Heart Failure | 1 1 | ${\sim}_{2} ( {c}_{i , k} , {c}_{j , k} ) = \{ \begin{matrix}1 , & if {c}_{i , k} = {c}_{j , k} \\ 0 , & otherwise\end{matrix}$ |
| COPD | 1 | If the precompiled model’s patient had the same existing |
| Hypertension | 1 | condition as the current patient the value is 1; otherwise, |
| | | the value is 0. |
| Other conditions . . . | . . . | The more patients in the precompiled model with the |
| | | same existing condition as the current patient the higher |
| | | the existing condition similarity score will be. |

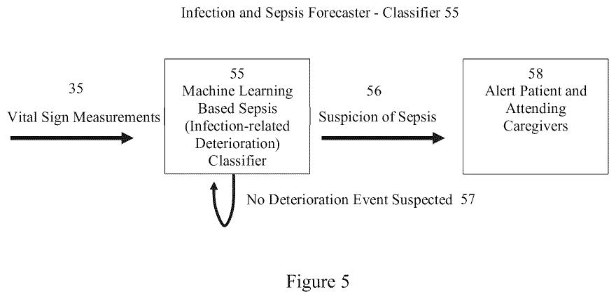

FIG. 5 shows the data flow of the sepsis prediction model which in this embodiment is a machine learning based classifier component 55 within the infection and sepsis forecaster 50 for this lower-level example. The classifier is merely continually receiving vital signs data 45 from the wearable devices on the patient. It is applying the parameters (thresholds) of the current model to this data stream to identify infection related deterioration events. If the classifier finds/determines that a deterioration event (sepsis onset) is likely 56, it can alert the patient and the caregivers to this fact 58. If it does not believe that a deterioration due to infection is likely 57, then it can simply continue monitoring. The model updated/reselection component described above will change the model (i.e. select a new model from model store 20) as appropriate if the patient's symptoms are to change. The alerts given to the patient and caregiver can provide as much or as little information as each decides is necessary (this can be preconfigured). The patient could simply be told that his/her infection may get worse and advise treatment is likely necessary. The caregiver or clinician could be given information about why the model believes the patient's condition might deteriorate (what features in the vital signs are predicting that) as well as information about the “like patients” that compose the model that is making this prediction. The clinician/caregivers can provide feedback to the architecture at the time of alert as well as when additional testing has been completed to allow for the system to update its prediction as well as use the current patient in future monitoring (i.e. provide outcome data on an event which can be used in training).

Implementation of the system and method disclosed herein for predicting infection and sepsis on a more personalized basis can combine both hardware and software elements. The hardware elements involved for patient measurements 32 may be a variety of wearable, home, and community based biomedical sensors. This may include, but is not limited to, analog and digital sensors, invasive and non-invasive devices, consumer and medical grade devices, wearable sensors of any form factors such as arm band, wrist bands, watches, adhesive sensors, and biosensors of any sensing modalities such as electrical, optical and thermal, audio, radio systems. The consumer or medical wearable devices or monitors employed are, in one example, embedded systems comprised of battery or power supply, timers, microprocessor unit, volatile or non-volatile memory units including read-only memory, random access memory, distributed memory storages, and secured memory cards or cartridges, user interfaces, communication ports and circuitries, transceivers, and digital display units. The patient and clinician interface hardware elements of embodiments of the system may include, but not limited to, mobile smartphones, tablets, bedside monitors, wall mounted relays, digital displays, Internet of Things (IoT), edge computing devices, remote data centers and servers.

Example embodiments of the method disclosed herein may also comprise software elements including, but not limited to, a firmware library, an application software or application programmable interface. The optimized prediction model for infection and sepsis forecaster 50 can be deployed as a mobile application programming interface (API) or application software or a web API or application software running on a web browser of a computing device. In one example, the algorithm workflow for detecting infection and sepsis and prompting for clinical actions can be deployed into the cloud computing of data centers, where the training or learning of configured prediction model can take place on any of the remote cloud services.

Embodiments of the system may be implemented using a computing apparatus, including distributed computing and cloud-based apparatus, and comprising one or more processors 70 and one or more memories 71. In some embodiments the one or more processor 70 may comprise one or more Central Processing Units (CPUs) or Graphical processing units (GPU) configured to perform some of the steps of the methods. A CPU may comprise an Input/Output Interface, an Arithmetic and Logic Unit (ALU) and a Control Unit and Program Counter element which is in communication with input and output devices through the Input/Output Interface. The Input/Output Interface may comprise a network interface and/or communications module for communicating with an equivalent communications module in another device using a predefined communications protocol (e.g. IEEE 802.11, IEEE 802.15, 4G/5G, TCP/IP, UDP, etc.). The computing apparatus may comprise a single CPU (core) or multiple CPUs (multiple core), or multiple processors. The computing apparatus is typically a cloud-based computing apparatus using GPU clusters, but may be a parallel processor, a vector processor, or be a distributed computing device. Memory is operatively coupled to the processor(s) and may comprise RAM and ROM components and may be provided within or external to the device or processor module. The memory may be used to store an operating system and additional software modules or instructions. The processor(s) may be configured to load and executed the software modules or instructions stored in the memory.

Software modules, also known as computer programs, computer codes, or instructions, may contain a number a number of source code or object code segments or instructions, and may reside in any computer readable medium such as a RAM memory, flash memory, ROM memory, EPROM memory, registers, hard disk, a removable disk, a CD-ROM, a DVD-ROM, a Blu-ray disc, or any other form of computer readable medium. In some aspects the computer-readable media may comprise non-transitory computer-readable media (e.g., tangible media). In addition, for other aspects computer-readable media may comprise transitory computer-readable media (e.g., a signal). Combinations of the above should also be included within the scope of computer-readable media. In another aspect, the computer readable medium may be integral to the processor. The processor and the computer readable medium may reside in an ASIC or related device. The software codes may be stored in a memory unit and the processor may be configured to execute them. The memory unit may be implemented within the processor or external to the processor, in which case it can be communicatively coupled to the processor via various means as is known in the art.

The mobile, desktop and web applications may be developed and built using a high-level language such as C++, JAVA, etc. including the use of toolkits such as Qt. Embodiments of the method use machine learning to build a model such as a classifier (or classifiers) using reference data sets including test and training sets. The term machine learning will be used to broadly to cover a range of algorithms/methods/techniques including supervised learning methods and Artificial Intelligence (AI) methods including convolutional neural nets and deep learning methods using multiple layered classifiers and/or multiple neural nets. The classifiers may use various data processing and statistical techniques such as feature extraction, detection/segmentation, mathematical morphology methods, digital image processing, objection recognition, feature vectors, etc. to build up the classifier. Various algorithms may be used including linear classifiers, regression algorithms, support vector machines, neural networks, Bayesian networks, etc. Computer vision or image processing libraries provide functions which can be used to build a classifier such as Computer Vision System Toolbox, MATLAB libraries, OpenCV C++ Libraries, ccv C++ CV Libraries, or ImageJ Java CV libraries and machine learning libraries such as Tensorflow, Caffe, Keras, PyTorch, deeplearn, Theano, etc.

Machine learning and Artificial Intelligence covers a range of algorithms. These algorithms include supervised classifiers, which find patterns in labelled training data that are indicative of a certain entity belonging to a certain class. Here, labelled indicates that for a set of patients the class to which they belong is known. For example, supervised classifiers could find indicators of whether or not a patient has sepsis based upon features extracted from the training data. The training data may be, for instance, patients' vital signs. This is done by exploring different weightings for different combinations of features. The resulting trained model mathematically captures the best or most accurate pattern for placing the entities in the training set into one of multiple (potentially many) classes. These features can be derived by researchers or automatically derived using algorithms known in the art. The weight of these features that best divide the training data can then be applied to patients for which the correct class is unknown to predict which class the unknown entity fits in.

Machine learning includes supervised machine learning or simply supervised learning methods which learns patterns in labelled training data. During training the labels or annotations for each data point (image) relates to a set of classes in order to create a predictive model or classifier that can be used to classify new unseen data. A range of supervised learning methods may be used including Random Forest, Support Vector Machines, decision tree, neural networks, k-nearest neighbors, linear discriminant analysis, naïve Bayes, and regression methods. Typically, a set of feature descriptors are extracted (or calculated) from a dataset or image (for example using a computer vision or image processing libraries) and the machine learning method are trained to identify the key features of the data items dataset which can be used to distinguish and thus classify new data. During machine learning training, models are built using different combinations of features to find a model that successfully classifies input data.

Deep learning is a form of machine learning/AI that has been developed to imitate the function of a human neural system. Deep learning models typically consist of artificial “neural networks”, typically convolutional neural networks that contain numerous intermediate layers between input and output, where each layer is considered a sub-model, each providing a different interpretation of the data. In contrast to many machine learning classification methods which calculate and use a (defined) set of feature descriptors and labels during training, deep learning methods ‘learn’ feature representations from the input data which can then be used to identify features or objects from other unknown datasets. That is a raw data is sent through the deep learning network, layer by layer, and each layer would learn to define specific (numeric) features or combinations of the input data items which can be used to classify the data. A variety of deep learning models are available each with different architectures (i.e. different number of layers and connections between layers) such as residual networks (e.g. ResNet-18, ResNet-50 and ResNet-101), densely connected networks (e.g. DenseNet-121 and DenseNet-161), and other variations (e.g. Inception V4 and Inception-ResNetV2). Training involves trying different combinations of model parameters and hyper-parameters, including input image resolution, choice of optimizer, learning rate value and scheduling, momentum value, dropout, and initialization of the weights (pre-training). A loss function may be defined to assess performances of a model, and during training a Deep Learning model is optimised by varying learning rates to drive the update mechanism for the network's weight parameters to minimize an objective/loss function.

Training of a machine learning classifier typically comprises:
a) Obtaining a dataset along with associated classification labels (e.g. outcomes);
b) Pre-processing the data, which includes data quality techniques/data cleaning to remove any label noise or bad data and preparing the data so it is ready to be utilised for training and validation;
c) Extract features or a set of feature descriptors (this may be omitted or performed during training, or the model may choose which features to use to classify the dataset);
d) Choosing a model configuration, including model type/architecture and machine learning hyper-parameters;
e) Splitting the dataset into a training dataset and a validation dataset and/or a test dataset;
f) Training the model by using a machine learning algorithm (including using neural network and deep learning algorithm) on the training dataset; typically, during the training process, many models are produced by adjusting and tuning the model configurations in order to optimise the performance of model according to an accuracy metric; and
g) Choosing the best “final” model based on the model's performance on the validation dataset; the model is then applied to the “unseen” test dataset to validate the performance of the final machine learning model.

Typically, accuracy is assessed by calculating the total number of correctly identified events in each category, divided by the total number of events, using a blind test set. Numerous variations on the above training methodology or the performance measures may be used as would be apparent to the person of skill in the art. For example, in some embodiments only a validation and test dataset may be used in which the dataset is trained on a training dataset, and the resultant model applied to a test dataset to assess accuracy. In other cases, training the machine learning classifier may comprise a plurality of Train-Validate Cycles. The training data is pre-processed and split into batches (the amount of data in each batch is a free model parameter but controls how fast and how stably the algorithm learns). After each batch, the weights of the network are adjusted, and the running total accuracy so far is assessed. In some embodiment weights are updated during the batch for example using gradient accumulation. When all patients have been assessed, one Epoch has been carried out, and the training set is shuffled (i.e. a new randomisation with the set is obtained), and the training starts again from the top, for the next epoch. During training a number of epochs may be run, depending on the size of the data set, the complexity of the data and the complexity of the model being trained. After each epoch, the model is run on the validation set, without any training taking place, to provide a measure of the progress in how accurate the model is, and to guide the user whether more epochs should be run, or if more epochs will result in overtraining. The validation set guides the choice of the overall model parameters, or hyperparameters, and is therefore not a truly blind set. Thus, at the end of the training the accuracy of the model may be assessed on a blind test dataset.

Once a model is trained it may be exported as an electronic data file comprising a series of model weights and associated data (e.g. model type) and stored in the data store 20. During deployment the model data file can then be loaded to configure a machine learning classifier to classify data in the infection and sepsis forecaster 50.

Sepsis prediction is a hard task. The timing of sepsis diagnosis is of the upmost importance; it profoundly affects the clinical outcomes of patients as well as healthcare utilization and costs. Treatment becomes increasingly expensive by the severity level of sepsis and when sepsis is not diagnosed at admission or when the patient's condition deteriorates. Therefore, accurate and reliable early diagnosis of sepsis are critical to lower sepsis related mortality and healthcare costs. Early detection of infection and sepsis is particularly crucial for timely intervention with optimal medical therapies when managing patients with chronic complex medical conditions such as heart failure who are prone to infection and sepsis development. A major proportion of sepsis cases originate outside the hospital settings where monitoring of vital signs and collecting patient inputs on symptoms and outcomes are recently evolving. Even with these advances, it remains very challenging to detect and intervene in advance of deterioration due to sepsis in out-of-hospital care environments. The incidences of sepsis related in-hospital deaths are substantial, and the associated healthcare costs are staggering. Marked contributors to these statistics are patient cases that were caught late.

Many proposed systems are inherently unsuitable as they use features to predict sepsis that are only available in clinical environments, where the patient is already under close supervision, are unrealistic to collect in settings where sepsis development can be most prevalent. In contrast embodiments of the present method use measurements, such as vital signs, which are accessible outside of these clinical environments, and importantly do have predictive power. The use of models trained on features available outside of clinical environments can thus be used to help patients and caregivers ensure earlier and more successful interventions for infections.

Another issue with many proposed systems is that the populations that are used to evaluate machine learning models and can be manipulated in offline analysis to skew the standard metrics found in the literature and thus misrepresent the practical use of such systems, i.e. by controlling choice of training population and metric they can inflate their efficacy and are not sufficiently robust for real world deployment. For example, populations with low incidence of sepsis can be used to validate a model and statistics that minimally punish false positives can be used to skew the practicality of a proposed system. In contrast embodiments of the system and method described herein employ pre-trained models selected on a best fitting homogeneous patient population (the most “like patients”) such as based on the similarity of the given patient measurements and health data records to the training population, to enable improved model precision and forecasting of infection and sepsis conditions and to further timely alert the clinicians and healthcare providers in case of positive events and patient deterioration.

Embodiments of the present system thus provide an online, adaptive, and practical platform for sepsis prediction. Embodiments of the method and system described herein may involve cascaded stages of input/output, processing and automated decision making for a real-time prospective forecasting of infection and sepsis that is applicable for any patient monitoring settings such as critical care, general hospital ward, out-of-hospital or home settings. Embodiments further describe how to effectively combine the patient interface and clinician interface, derived inputs and patient measurements, determine the current patient's similarity measure (or score), select a personalized or population based pretrained sepsis prediction model based on the patient similarity measure, forecast the infection or sepsis condition in advance, generate notifications to be displayed in clinician interface tools, update the Data Repository with clinician's inputs and adapt/retrain the predictive models from time-to-time.

As described herein a sophisticated solution for the early detection of sepsis has been described to overcome the current limitations of existing systems. The present disclosure describes embodiments of a personalized infection and sepsis detection system using convenient continuous vital sign monitoring, patient interfaces to input self-reporting of symptoms and signs, and clinician interfaces to input inference validations and for patient management. Embodiments of the methods and systems described herein thus enable personalized and precise prediction of sepsis through individualized and persistent patient monitoring. The system may be implemented as a fully integrated remote patient monitoring solution allowing real-time collection of various electronic health records, patient symptoms and physiological and activity parameters, alerting of caregivers and inputting of clinical decisions etc. In addition to identifying models trained using the most similar patient cohort, the selection of the most similar patient cohort, and associated model is updated (that is the best model is reselected) based on the trajectory of infection progression or sepsis development and associated clinical parameters. That is the models can be adapted (personalized) after receiving additional data from a patient or the patient's caregiver, depending on the data that is available. Additionally as new data and patients are obtained, the models may be retrained.

Those of skill in the art would understand that information and signals may be represented using any of a variety of technologies and techniques. For example, data, instructions, commands, information, signals, bits, symbols, and chips may be referenced throughout the above description may be represented by voltages, currents, electromagnetic waves, magnetic fields or particles, optical fields or particles, or any combination thereof.

Those of skill in the art would further appreciate that the various illustrative logical blocks, modules, circuits, and algorithm steps described in connection with the embodiments disclosed herein may be implemented as electronic hardware, computer software or instructions, middleware, platforms, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The steps of a method or algorithm described in connection with the embodiments disclosed herein may be embodied directly in hardware, in a software module executed by a processor, or in a combination of the two, including cloud based systems. For a hardware implementation, processing may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, or other electronic units designed to perform the functions described herein, or a combination thereof. Various middleware and computing platforms may be used.

Further, it should be appreciated that modules and/or other appropriate means for performing the methods and techniques described herein can be downloaded and/or otherwise obtained by computing apparatus. For example, such an apparatus can be coupled to a server to facilitate the transfer of means for performing the methods described herein. Alternatively, various methods described herein can be provided via storage means (e.g., RAM, ROM, a physical storage medium such as a compact disc (CD) or floppy disk, etc.), such that a computing device can obtain the various methods upon coupling or providing the storage means to the device. Moreover, any other suitable technique for providing the methods and techniques described herein to a device can be utilized.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

It will be understood that the terms “comprise” and “include” and any of their derivatives (e.g. comprises, comprising, includes, including) as used in this specification is to be taken to be inclusive of features to which the term refers, and is not meant to exclude the presence of any additional features unless otherwise stated or implied

The reference to any prior art in this specification is not, and should not be taken as, an acknowledgement of any form of suggestion that such prior art forms part of the common general knowledge.

It will be appreciated by those skilled in the art that the disclosure is not restricted in its use to the particular application or applications described. Neither is the present disclosure restricted in its preferred embodiment with regard to the particular elements and/or features described or depicted herein. It will be appreciated that the disclosure is not limited to the embodiment or embodiments disclosed, but is capable of numerous rearrangements, modifications and substitutions without departing from the scope as set forth and defined by the following claims.

1. A computational method for detection of infection and sepsis, the method comprising:
storing patient health data in a data store for a plurality of patients, the patient health data for a patient comprising a plurality of data items comprising a plurality of clinical data items obtained from one or more clinical data sources, a plurality of patient measurement data obtained from one or more wearable, home, and community based biomedical sensors, and a plurality of symptoms obtained from the patient;
generating and storing a plurality of sepsis prediction models and a general population sepsis prediction model each trained using the stored patient health data wherein the general population sepsis prediction model is generated by training a sepsis prediction model on a general population of patients drawn from the plurality of patients and generating each of the plurality of sepsis prediction models comprises:
identifying a training cohort of similar patients according to a patient similarity measure wherein each patient similarity measure is determined using a different combination of data items in the patient health data, one or more similarity functions and/or one or more similarity criterion; and
training a sepsis prediction model using the training cohort of similar patients;
obtaining patient health data for a monitored patient, the patient health data comprising a plurality of clinical data items obtained from one or more clinical data sources, a plurality of patient measurement data obtained from one or more wearable, home, and community based biomedical sensors, and a plurality of symptoms obtained from the patient;
selecting a sepsis prediction model from the plurality of sepsis prediction models for monitoring the monitored patient, where the sepsis prediction model is selected by identifying the sepsis prediction model with the training cohort most similar to the monitored patient, and if no similar training cohort can be identified then selecting the general population sepsis prediction model;
using the selected sepsis prediction model to monitor the monitored patient to detect infection and sepsis events, and generating electronic alerts if an infection and sepsis event is detected;
repeating the selecting step in response to a change in the patient health data of the monitored patient over time; and
repeating the generating and storing step in response to one or more confirmations of detected infection and sepsis events.

2. The method as claimed in claim 1, wherein the one or more clinical data sources comprises electronic medical records and a clinician user interface configured to receive clinical notes from a clinician.

3. The method as claimed in claim 1, wherein one or more of the plurality of patient measurement data obtained from the monitored patient comprises repeated measurements of one or more vital signs, with each measurement having an associated time.

4. The method as claimed in claim 1, wherein the one or more personal, home, and community based biomedical sensors comprise one or more wearable sensors and vital sign sensors.

5. The method as claimed in claim 1, wherein one or more of the plurality of patient symptoms obtained from the monitored patient are obtained and entered using a patient user interface executing on a mobile computing apparatus.

6. The method as claimed in claim 1, wherein each sepsis prediction model is a machine learning classifier which is configured to monitor updates to patient health data for the monitored patient and generate an alert if an infection and sepsis event is detected.

7. The method as claimed in a claim 1, wherein identifying the sepsis prediction model with the training cohort most similar to the monitored patient comprises:
filtering the plurality of sepsis prediction models to identify a set of similar models based on one or more current symptoms, one or more current disease conditions and one or more current vital signs of the monitored patients; and
selecting a model from the set of similar models based on the model in the set of similar models in which the training cohort of patients have the most similar set of medical conditions to the monitored patient.

8. The method as claimed in claim 7, wherein
filtering the plurality of sepsis prediction models is performed by calculating a similarity score for each model which is a weighted sum of the of the similarity between one or more current symptoms, one or more current disease conditions and one or more current vital sign measurements of the monitored patient, and the corresponding one or more symptoms and one or more vital sign measurements of patients in the training cohort of the respective model; and
selecting a model from the set of similar models is performed by calculating, for each model in the set of similar scores, a total similarity score by, for one or more medical conditions, determining a similarity score between the medical condition of the monitored patient and corresponding medical condition for each patient in the training cohort of the respective model and multiplying the similarity score by a weight for the medical condition, and then summing each of the weighted similarity scores to obtain the total similarity score; and selecting the model with the highest total similarity score.

9. The method as claimed in claim 1, further comprising storing a plurality of trigger conditions and repeating the selecting step is performed in response to an update in patient health data satisfying one or more of the trigger conditions.

10. The method as claimed in claim 1, wherein the electronic alert comprises an alert to a clinician via a clinician user interface, and wherein the clinician user interface is configured to allow the clinician to confirm the validity of the infection and sepsis event, and one or more confirmations are used to trigger repeating the generating and storing step.

11. A computational apparatus configured for the detection of infection and sepsis in a monitored patient, the apparatus comprising:
one or more processors;
one or more memories operatively associated with the one or more processors;
a data store configured to store patient health data for a plurality of patients, the patient health data for a patient comprising a plurality of data items comprising a plurality of clinical data items obtained from one or more clinical data sources, a plurality of patient measurement data obtained from one or more wearable, home, and community based biomedical sensors, and a plurality of symptoms obtained from the patient via a patient user interface;
wherein the one or more memories comprise instructions to configure the one or more processors to:
generate and store in a model store a plurality of sepsis prediction models and a general population sepsis prediction model each trained using the stored patient health data obtained from the data store, wherein the general population sepsis prediction model is generated by training a sepsis prediction model on a general population of patients drawn from the plurality of patients and generating each of the plurality of sepsis prediction models comprises:
identifying a training cohort of similar patients according to a patient similarity measure wherein each patient similarity measure is determined using a different combination of data items in the patient health data, one or more similarity functions and/or one or more similarity criterion; and
training a sepsis prediction model using the training cohort of similar patients;
obtain patient health data for a monitored patient, the patient health data comprising a plurality of clinical data items obtained from one or more clinical data sources, a plurality of patient measurement data obtained from one or more wearable, home, and community based biomedical sensors, and a plurality of symptoms obtained from the patient;
select a sepsis prediction model from the plurality of sepsis prediction models for monitoring the monitored patient, where the sepsis prediction model is selected by identifying the sepsis prediction model with the training cohort most similar to the monitored patient, and if no similar training cohort can be identified then selecting the general population sepsis prediction model;
use the selected sepsis prediction model to monitor the monitored patient to detect infection and sepsis events, and generating electronic alerts if an infection and sepsis event is detected;
repeat the selecting step in response to a change in the patient health data of the monitored patient over time; and
repeat the generating and storing step in response to one or more confirmations of detected infection and sepsis events.

12. The computational apparatus as claimed in claim 11, wherein the one or more memories further comprise instructions to further configure the one or more processors to provide a clinician user interface wherein the one or more clinical data sources comprises electronic medical records and the clinician user interface is configured to receive clinical notes from a clinician.

13. The computational apparatus as claimed in claim 11, wherein one or more of the plurality of patient measurement data obtained from the monitored patient comprises repeated measurements of one or more vital signs, with each measurement having an associated time.

14. The computational apparatus as claimed in any claim 11, wherein the one or more personal, home, and community based biomedical sensors comprise one or more wearable sensors and vital sign sensors.

15. The computational apparatus as claimed in any-e claim 11, wherein one or more of the plurality of patient symptoms obtained from the monitored patient are obtained and entered using a patient user interface executing on a mobile computing apparatus.

16. The computational apparatus as claimed in any-one claim 11, wherein each sepsis prediction model is a machine learning classifier which is configured to monitor updates to patient health data for the monitored patient and generate an alert if an infection and sepsis event is detected.

17. The computational apparatus as claimed in any-one claim 11, wherein identifying the sepsis prediction model with the training cohort most similar to the monitored patient comprises:
filtering the plurality of sepsis prediction models to identify a set of similar models based on one or more current symptoms and one or more current vital signs of the monitored patients; and
selecting a model from the set of similar models based on the model in the set of similar models in which the training cohort of patients have the most similar set of medical conditions to the monitored patient.

18. The computational apparatus as claimed in claim 17, wherein
filtering the plurality of sepsis prediction models is performed by calculating a similarity score for each model which is a weighted sum of the of the similarity between one or more current symptoms and one or more current vital sign measurements of the monitored patient, and the corresponding one or more symptoms and one or more vital sign measurements of patients in the training cohort of the respective model; and
selecting a model from the set of similar models is performed by calculating, for each model in the set of similar scores, a total similarity score by, for one or more medical conditions, determining a similarity score between the medical condition of the monitored patient and corresponding medical condition for each patient in the training cohort of the respective model and multiplying the similarity score by a weight for the medical condition, and then summing each of the weighted similarity scores to obtain the total similarity score; and selecting the model with the highest total similarity score.

19. The computational apparatus as claimed in #claim 11, wherein the one or more memories are further configured to store a plurality of trigger conditions and repeating the selecting step is performed in response to an update in patient health data satisfying one or more of the trigger conditions.

20. The computational apparatus as claimed in any one claim 11, wherein the one or more memories further comprise instructions to further configure the one or more processors to provide a clinician user interface, wherein the electronic alert comprises an alert sent to a clinician via the clinician user interface, and the clinician user interface is configured to allow the clinician to confirm the validity of the infection and sepsis event, and one or more confirmations are used to trigger repeating the generating and storing step.
